(19) United States
(12) Patent Application Publication
(10) Pub. No.: US 2022/0215958 A1
(43) Pub. Date: Jul. 7, 2022

(54) SYSTEM AND METHOD FOR TRAINING MACHINE LEARNING MODELS WITH UNLABELED OR WEAKLY-LABELED DATA AND APPLYING THE SAME FOR PHYSIOLOGICAL ANALYSIS

(71) Applicant: SHENZHEN KEYA MEDICAL TECHNOLOGY CORPORATION, Shenzhen (CN)

(72) Inventors: Bin Kong, Charlotte, NC (US); Youbing Yin, Kenmore, WA (US); Xin Wang, Seattle, WA (US); Yi Lu, Seattle, WA (US); Haoyu Yang, Seattle, WA (US); Junjie Bai, Seattle, WA (US); Qi Song, Seattle, WA (US)

(73) Assignee: SHENZHEN KEYA MEDICAL TECHNOLOGY CORPORATION, Shenzhen (CN)

(21) Appl. No.: 17/568,084

(22) Filed: Jan. 4, 2022

Related U.S. Application Data
(60) Provisional application No. 63/133,756, filed on Jan. 4, 2021.

(51) Int. Cl. G16H 50/20; G16H 30/40; G06T 7/00; G06N 20/20

(52) U.S. Cl. CPC G16H 50/20; G16H 30/40; G06T 2207/30096; G06N 20/20; G06T 7/0012

(57) ABSTRACT

The present disclosure relates to training methods for a machine learning model for physiological analysis. The training method may include receiving training data including a first dataset of labeled data of a physiological-related parameter and a second dataset of weakly-labeled data of the physiological-related parameter. The training method further includes training, by at least one processor, an initial machine learning model using the first dataset, and applying, by the at least one processor, the initial machine learning model to the second dataset to generate a third dataset of pseudo-labeled data of the physiological-related parameter. The training method also includes training, by the at least one processor, the machine learning model based on the first dataset and the third dataset, and providing the trained machine learning model for predicting the physiological-related parameter. Thereby, the weakly-labeled dataset may be sufficiently utilized in training of the machine learning model and improve ts p iformance.

### CROSS REFERENCE TO RELATED APPLICATION

This application is based on and claims the benefit of priority of U,S, Provisional Application No. 63/133,756, filed on Jan. 4, 2021, which is incorporated herein by reference in its entirety.

### TECHNICAL FIELD

The present disclosure relates to technical field of processing and analysis for medical data and medical images, more specifically, to technical field of training machine learning model based on unlabeled or weakly-labeled data and applying the trained machine learning model for physiological-related parameter prediction.

### BACKGROUND

Recent advances in machine learning make it possible to model extremely complex functions. For instance, a deep learning system can accurately categorize an image, even outperforming human annotators, However, one of the challenges with such complex models is that they require large-scale dataset with high quality labels. In the field of healthcare, a small amount of labeled data is often available for training machine learning models. As a result, the trained model is very likely to overfit the training data, which makes it difficult to generalize to unseen test data.

For example, fractional flow reserve (FFR) or instantaneous wave-free ratio (iFR) is considered as a reference standard in evaluating hemodynamics significance of stenosis for coronary artery diseases. Attempts have been made to estimate FFR or other quantitative measurements using image data such as computed tomography (CT), However, it requires invasive surgeries to get FFR measurements; making it very challenging to build a large scale of training data for such image based FFR prediction tasks. Additionally, high-quality annotations of medical data have to be performed by experts with specialized trainings in the domain. The high dimensionality of medical data also makes annotation time-consuming. For example, a whole-slide image with 20,000×20,000 pixels for a lymph node section equires significant amount of time from board-certified experts for annotation.

Numerous approaches have been proposed to address the overfitting problems of machine learning models. For instance, early stopping (a learning procedure is terminated earlier when a criterion is reached) is often used to avoid overfitting the noises in the training data. However, this approach ignores the challenges imposed by weakly or unlabeled data to the regularization in the field of healthcare.

Some conventional methods may consider regularizing machine learning models by post-processing steps. However, such methods require additional steps and may decrease the performance of the machine learning models. Some other methods may use one or more loss term(s) to penalize incorrect prediction in the training stage, hoping to obtain a more regularized and robust machine learning model. However, these methods do not address the fundamental problem of lack of training data, and pay little attention to the application of weakly-labeled data containing the unlabeled data therein in training.

### SUMMARY

The disclosure is provided to solve the above issues existing in the prior art.

The present disclosure provides a training method and system of a machine learning model for physiological-related parameter prediction and non-transitory computer-readable storage medium for the same, The disclosed method and system leverage the weakly-labeled data, which are easier to obtain compared to high quality labeled data, to enable the machine learning model to learn better data representations. Accordingly, the disclosed method and system can improve the performance of the machine learning model, including the prediction accuracy, the robustness and the generalization ability of the machine learning model.

According to a first aspect of the present disclosure, it provides a training method for a machine learning model for physiological analysis. The training method may include receiving training data including a first dataset of labeled data of a physiological-related parameter and a second dataset of weakly-labeled data of the physiological-related parameter. The training method further includes training, by at least one processor, an initial machine learning model using the first dataset, and applying, by the at least one processor, the initial machine learning model to the second dataset to generate a third dataset of pseudo-labeled data of the physiological-related parameter. The training method also includes training, by the at least one processor, the machine learning model based on the first dataset and the third dataset, and. providing the trained machine learning model for predicting the physiological-related parameter, The present disclosure also provides a system for training the machine learning model using the above method and a non-transitory computer-readable medium storing computer instructions that can be executed by at least one processor to perform the above method.

According to a second aspect of the present disclosure, it provides a training method for a machine learning model for physiological analysis. The training method may include receiving training data comprising weakly-labeled data of a physiological-related parameter. The training method further includes performing, by at least one processor, a first transformation on the weakly-labeled data to form a first transformed dataset, and performing, by the at least one processor, a second transformation on the weakly-labeled data to form a second transformed dataset. The training method also includes training, by the at least one processor, the machine learning model based on the training data, the first transformed dataset and the second transformed dataset. The training minimizes a difference between a first prediction result of the physiological-related parameter obtained by applying the machine learning model to the first transformed dataset and a second prediction result of the physiological-related parameter obtained by applying the machine learning model to the second transformed dataset. The training method additionally includes providing the trained machine learning model for predicting the physiological-related parameter. The present disclosure also provides a system for training the machine learning model using the above method and a non-transitory computer-readable medium storing computer instructions that can be executed by at least one processor to perform the above method.

According to a third aspect of the present disclosure, it provides a training method for a machine learning model for physiological analysis. The training method includes receiving training data comprising weakly-labeled data of a physiological-related parameter. The training method further includes training, by at least one processor, the machine learning model with an ensembled model based on the training data. The machine learning model has a first set of model parameters and the ensembled model has a second set of model parameters derived from the first set of model parameters. The training minimizes a difference between a first prediction result of the physiological-related parameter obtained by applying the machine learning model to the weakly-labeled data and a second prediction result of the physiological-related parameter obtained by applying the ensembled model to weakly-labeled data. The training method also includes providing the trained machine learning model for predicting the physiological-related parameter. The present disclosure also provides a system for training the machine learning model using the above method and a non-transitory computer-readable medium storing computer instructions that can be executed by at least one processor o perform the above method.

The training method and system of a machine learning model for physiological analysis (such as physiological-related parameter prediction) and storage medium according to each embodiment of the present disclosure may leverage prior information of the weakly-labeled data to augment and supplement the labels in the weakly-labeled data during training of the machine learning model. The trained machine learning model has an improved accuracy of physiological-related parameter prediction, as well as improved robustness and generalization ability.

The foregoing general description and the following detailed description are only exemplary and illustrative, and do not intend to limit the claimed invention.

### BRIEF DESCRIPTION OF THE DRAWINGS

In the drawings, which are not necessarily drawn to scale, like reference numerals may describe similar components in different views. Like reference numerals having letter suffixes or different letter suffixes may represent different instances of similar components. The drawings illustrate generally, by way of example, but not by way of limitation, various embodiments, and together with the description and claims, serve to explain the disclosed errtbodiments. Such embodiments are demonstrative and not intended to be exhaustive or exclusive embodiments of the present method, device, system, or non-transitory computer readable medium having instructions thereon for implementing the method.

FIG. 1 illustrates a flowchart of a first exemplary training method of a machine learning model for physiological-related parameter prediction, according to an embodiment of the present disclosure.

FIG. 2 illustrates a schematic diagram of the first exemplary training method of FIG. 1, according to the embodiment of the present disclosure.

FIG. 3 illustrates a flowchart of a second exemplary training method of a machine learning model for physiological-related parameter prediction, according to an embodiment of the present disclosure.

FIG. 4 illustrates a schematic diagram of the second exemplary training method of FIG. 3, according to the embodiment of the present disclosure.

FIG. 5 illustrates a flowchart of a third training method of a machine learning model for physiological-related parameter prediction, according to an embodiment of the present disclosure.

FIG. 6 illustrates a schematic diagram of the second exemplary training method of FIG. 2, according to the embodiment of the present disclosure.

FIG. 7 illustrates a flowchart of the training and testing processes of physiological-related parameter prediction by using training data including labeled data and weakly-labeled data, according to an embodiment of the present disclosure.

FIG. 8 illustrates a schematic block diagram of a training system of the machine learning model for physiological-related parameter prediction, according to the embodiment of the present disclosure.

### DETAILED :DESCRWITON

Reference in details will be made to the exemplary embodiment herein, examples of which illustrates in accompany drawings. In the present disclosure, the physiological-related parameter may indicate at least one of physiological functional state, blood pressure, blood velocity, blood flow-rate, wall-surface shear stress, fractional flow reserve (FFR), microcirculation resistance index (IMR), and instantaneous wave-free ratio (iFR) and/or a combination thereof. In some embodiments, it may be used to qualitatively indicate specific conditions, such as lesion or sub-health condition in a tissue and a vessel, etc. and it may also be a value to quantitatively indicate specific conditions, such as FFR value of the vessel, etc. However, the physiological-related para -ter in the present disclosure is not limited to this, and it may be any features, parameters and conditions and so on that are needed in clinical medicine and may be predicted and identified by data processing or image analysis. In present disclosure, a machine learning model can include any learning model that may learn through a training process based on training dataset, such as but not limited to traditional learning model, deep learning model, or a combination thereof.

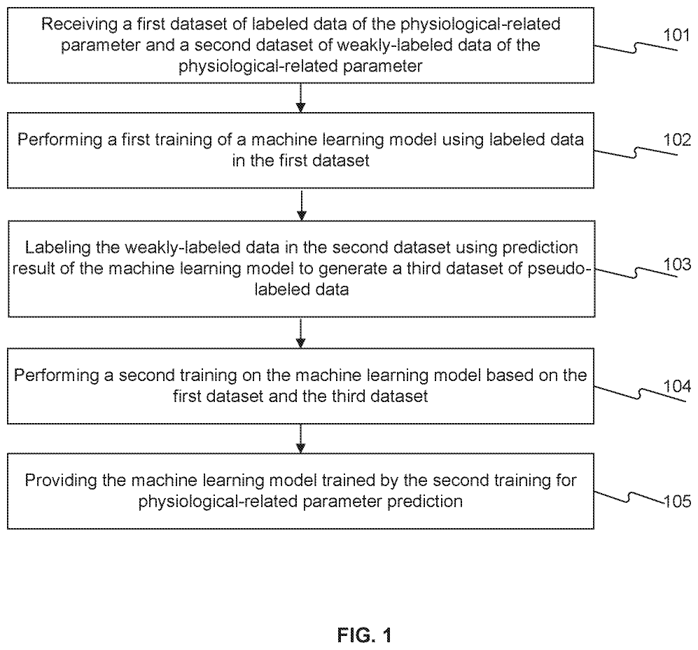

FIG. 1 illustrates a flowchart of a first exemplary training method of a machine learning model for physiological-related parameter prediction, according to an embodiment of the present disclosure.

In step 101, training data including both a first dataset of labeled data of the physiological-related parameter and a second dataset of weakly-labeled data of the physiological-related parameter may be received.

In some embodiments, the labeled data in the first dataset and the weakly-labeled data in the second dataset may include image data. The image data may include at least one of the following image data from plurality of data sources and/or a combination thereof: functional MRI, Cone Beam CT (CBCT), Spiral CT, Positron Emission Tomography (PET), Single-Photon Emission Computed Tomography (SPECT), X-ray, optical tomography, fluorescence imaging, ultrasound imaging, and radiotherapy portal imaging and so on. In other embodiments, the labeled data and unlabeled data of the physiological-related parameter may be also acquired from any other data source, limitations on which are not made by the present disclosure.

In the present disclosure, labeled data of a first dataset may include clean labeled data. The weakly-labeled dataset may include but may not be limited to noisy labeled data, partially labeled data, and unlabeled data.

In step 102, a first training of a machine learning model is performed using the labeled data in the first dataset. The machine learning model trained by this first training step may be referred to as the initial machine learning model.

In step 103, a process of label complement may be performed by applying the initially trained machine learning model from step 102 to the weakly-labeled data in the second dataset, to obtain a third dataset of pseudo-labeled data of the physiological-related parameter. In this step, the weakly-labeled data may be labeled or relabeled using the prediction result obtained by the initial machine learning model when applied to the weakly-labeled data.

In some embodiments, the label complement may include at least one of supplementation, cleaning or modification for label of data in the second dataset.

In some embodiments, the process of label complement may be further based on prior information associated with the weakly-labeled data. For example, when the physiological-related parameter is an FFR or an iFR, the prior information may include at least one of the following and/or a combination thereof: a label that a FFR value at ostia point is labeled as 1 or indicates a maximum value, data of a vessel without lesion is labeled to indicate normality value or normal label, data of a vessel with first stenosis degree or more severe stenosis is labeled to indicate functional significant value or label. In some alternative embodiments, label complement may be performed solely based on the prior information without using the initially trained machine learning model to predict the labels.

In step 104, a second training may be performed on the machine learning model based on the first dataset and the third dataset.

In some embodiments, before the third dataset is used for the second training of the machine learning model, it may be processed additionally in advance. For instance, a pseudo-labeled data satisfying a first preset condition associated with confidence level may be selected to be included in the third dataset if it meets the first preset condition. Pseudo-labeled data that does not satisfy the first preset condition may be considered as not suitable for training the machine learning model and thus is not included in the third dataset for subsequent training process. Accordingly, the second training may be performed on the machine learning model based on only the pseudo-labeled data satisfying the first preset condition, which are selected to be included in the third dataset.

In step 105, the machine learning model trained by the second training may be provided for physiological-related parameter prediction.

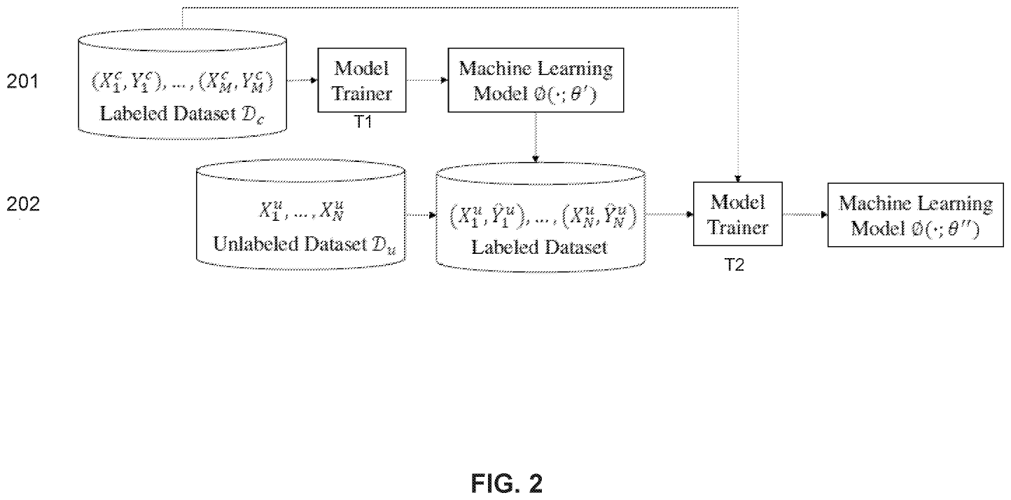

FIG. 2 illustrates schematic diagram of the first exemplary training method of FIG. 1, according to the embodiment of the present disclosure.

As shown in FIG. 2, assuming that a first dataset of labeled data of the physiological related parameter, that is, clean labeled dataset [symbol] _{c}, and a second dataset of weakly-labeled data of the physiological related parameterhas been received. Only as an example, the second dataset may be an unlabeled dataset [symbol] ᵤ herein. However, it is contemplated that the training method does not apply to unlabeled data, but may be adapted to other types of weakly-labeled data, such as partially unlabeled data, or noisy labeled data, etc.

Unlabeled dataset, partial labeled dataset or other kind of weakly-labeled dataset are oftentimes much easier to obtain, compared with well-annotated dataset, especially for domains that requires domain expertise such as healthcare. Usually, the size of the unlabeled dataset [symbol] ᵤ={X₁^{u}, . . . , X_{N}^{u}} may be at orders of magnitude much larger than that of the labeled dataset [symbol] _{c}={(X₁^{c}, Y₁^{c}), . . . , (X_{M}^{c}, Y_{M}^{c})}. Therefore, the effective leverage of the unlabeled dataset may boost the model performance, thereby the trained model may be used to generate more higher-quality predictions in the testing stage.

As shown in FIG. 2, in this embodiment, the leverage of weakly-labeled dataset [symbol] ᵤ for training machine learning model may be divided into two steps. Firstly, the first training is performed on the machine learning model, by the model trainer T1, based on the first dataset [symbol] _{c}, which is labeled dataset or said clean dataset, yielding a trained machine learning model Ø (·;θ′), as is illustrated in step 201 in top of FIG. 2. Ideally, if the machine learning model Ø (·; θ′) is well trained, the model can generalize to unseen test data and can generate reasonable predictions for these test data. Thus, in step 202, the data in unlabeled dataset [symbol] ᵤ may be used as unseen test data, and predictions may be performed using the trained machine learning model Ø(·; θ′) based on the unlabeled dataset [symbol] ᵤ, and the predicted labels may be used to complement labels for the unlabeled data Xₙ^{u} in [symbol] ᵤ, yielding a pseudo-labeled dataset [symbol] ᵤ={(X₁^{u}, Ŷ₁^{u}), . . . , (X_{N}^{u}, Ŷ_{N}^{u})}, that is, the third dataset. Finally, a second training may be performed by the model trainer T2 on the machine learning model Ø(·; θ′) based on the first dataset D, together with the third dataset [symbol] ᵤ, so as to generate the final learning model Ø(·; θ″), i.e., the machine learning model Ø(·; θ″) trained b the second training, which may be then used for subsequent physiological-related parameter prediction.

Using this example, in step 102, a regression model Ø (·; θ′) may be trained on the labeled dataset [symbol] _{c} using regression loss L_{c}, e.g., squared L₂ norm loss Σₘ₌₁^{M}∥Ŷₘ^{c}−Yₘ^{c}∥_{2′}² where Ŷₘ^{c}=Ø(Xₘ^{c}; θ′), and Yₘ^{c} is the ground truth value over Xₘ^{c}.

In step 103, Ø(·; θ′) may be used to label each data in the unlabeled dataset [symbol] ᵤ, yielding the pseudo-labeled dataset [symbol] ᵤ={(X₁^{u}, Ŷ₁^{u}), . . . , (X_{N}^{u}, Ŷ_{N}^{u})}, where Ŷₙ^{u}=Ø(Xₙ^{u}; θ′). In some embodiment, the pseudo-labels in [symbol] ᵤ may be noisy, and may be filtered with additional criteria, thereby the filtered pseudo-labeled data may be used in the following steps. In some embodiments, prior information associated with the unlabeled data in [symbol] ᵤ may be additionally or alternatively used to generate pseudo-labels of unlabeled data. For example, when the physiological-related parameter is FFR or iFR, the prior information may include at least one of the following and/or a combination thereof: a label that a FFR value at ostia point is labeled as 1 or indicates a maximum value, data of a vessel without lesion is labeled to indicate noiniality value or normal label, data of a vessel with first stenosis degree or more severe stenosis is labeled to indicate functional significant value or label. Any other applicable prior information may be applied to the process of pseudo-labeled data.

In step 104, the pseudo-labeled dataset [symbol] ᵤ may be used to calculate the additional regression loss term Lᵤ, e.g., squared L₂ norm loss Σₙ₌₁^{N}∥Ŷₙ^{u}∥₂², where Ŷₙ^{u}=Ø(Xₙ^{u}; θ′), and Ŷₙ^{u}=Ø(Xₙ^{u}; θ″).

In the above training method according to the embodiment of the present disclosure, with the pseudo-labeled dataset [symbol] ᵤ as additional labeled dataset, the second training may be performed using the additional regressiot. loss teitn Lᵤ, and yield a trained machine learning model ØO, which may be used as higher-quality model to perform higher-quality predictions in the testing stage.

In some other embodiments, after the pseudo-labeled dataset [symbol] ᵤ is generated, based on a first preset condition, the labeled data satisfying the first preset condition may be selected from the pseudo-labeled dataset [symbol] ᵤ. As an example, the first preset condition may at least be associated with confidence level, and the high-quality labeled data with high confidence level may be selected from the pseudo-labeled dataset [symbol] ᵤ and be used for the second training. Thus, the trained machine learning model after the second training will possess a better generalization ability as well as an improved accuracy when performing physiological-related parameter prediction.

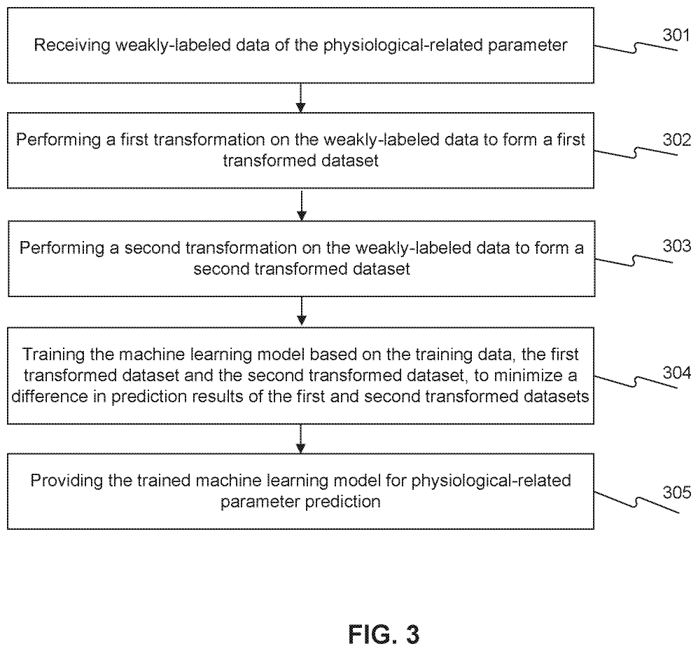

FIG. 3 illustrates a flowchart of a second exemplary training method of a machine learning model for physiological-related parameter prediction, according to an embodiment of the present disclosure.

In step 301, weakly-labeled data of the physiological-related parameter may be received. In some embodiments, the weakly-labeled data may include image data. The image data may include at least one of the following image data from plurality of data sources and/or a combination thereof; functional MRI, Cone Beam CT (CBCT), Spiral CT, Positron Emission Tomography (PET), Single-Photon Emission Computed Tomography (SPECT), X-ray, optical tomography, fluorescence imaging, ultrasound imaging, and radiotherapy portal imaging and so on. In other embodiments, weakly-labeled data of the physiological-related parameter may be also acquired from any other data source, limitations on which are not made by the present disclosure.

In the present disclosure the weakly-labeled dataset may include but may not be limited to noisy labeled data, partially labeled data, and unlabeled data.

In step 302, a first transformation is performed on the weakly-labeled data, or at least a subset of it, to form a first transfoiined dataset. Similarly, in step 303, a second transformation is performed on the same subset of weakly-labeled data to form a second transformed dataset. The first and second transformations are different transformation of the data. They can be selected, e.g., from rotation, translation and scaling of the data.

In step 304, the machine learning model is trained based on the received training data, and the first transformed dataset and the second transformed dataset. During the training, a difference in the prediction results of the first and second transformed datasets is minimized. The assumption is that prediction of an artificially transfoiined data example should be the same as the prediction of the original training example. Therefore, the prediction results of two different artificially transformed data should also be the same. More specifically, for a data example X′, two artificially transformation A and B are applied to it, yielding two transformed version X,¹¹₄ and X_{B}^{u}. Ideally,the predictions of X_{A}^{u} and X_{B}^{u}, i.e., Ŷ_{A}^{u} and Ŷ_{B}^{u} respectively, should be the same. This assumption is valid for many prediction problems, e.g., a rotated pathology image is still cancerous if the original image is cancerous.

In some embodiments, the training can be assisted by using a loss term formulated with the clean labeled data in the received training data. In yet some embodiments, the prior information derived from the weakly-labeled data may also be used to perform data labeling or generate additional loss item. For example, the prior information can be used to generate pseudo-labels for the unlabeled or weakly-labeled data. When the physiological-related parameter is an FFR or an iFR, the prior information may include at least one of the following and/or a combination thereof: a label that a FFR value at ostia point is labeled as 1 or indicates a maximum value, data of a vessel without lesion is labeled to indicate normality value or normal label, data of a vessel with first stenosis degree or more severe stenosis is labeled to indicate functional significant value or label.

In step 305, the trained machine learning model may be provided for physiological-related parameter prediction.

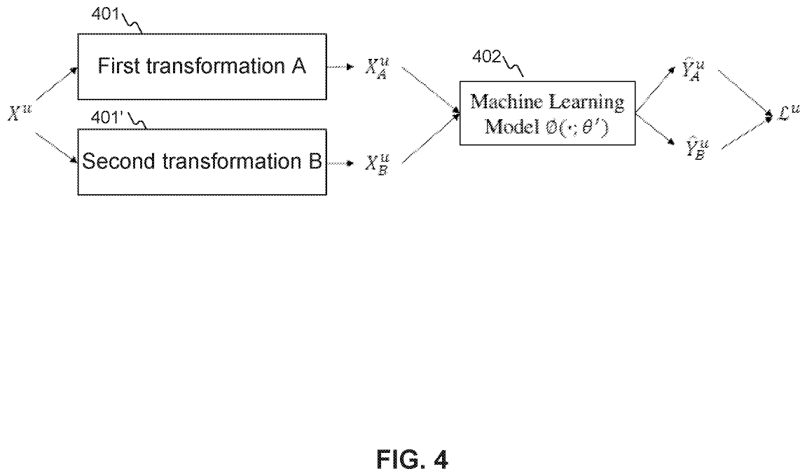

FIG. 4 illustrates a schematic diagram of the second exemplary training method of FIG. 3, according to the embodiment of the present disclosure.

In the example of FIG. 4, the unlabeled dataset [symbol] ᵤ is incorporated into the training procedure of the machine learning model by utilization of the property of the data, so that the trained model may be used to generate higher quality predictions in the testing stage.

For the convenience of description, it is assumed that the unlabeled dataset Dᵤ is an image dataset. It can be conceived that the prediction of an artificially transformed data example should be consistent with the prediction of the original training example. This assumption is valid for many prediction problems, e.g., the prediction result of a rotated or a translated pathology image is still cancerous if the prediction result of the original image is cancerous.

More specifically, for the data sample Xuof unlabeled dataset [symbol] ᵤ, a first transformation and a second transformation may be performed on Xu, in step 401 and step 401′ separately, and then the first transformed dataset and second transformed dataset, i.e., X_{A}^{u} and X_{B}^{u}, may be used for further training steps along with the training data, so as to obtain an augmented training dataset (not shown). In some embodiment, the first transformation and the second transformation are different transformations, and each may be selected from rotation, translation and scaling of an image and/or a combination thereof, but is not limited thereto.

Next, in step 402, a first prediction of physiological-related parameter on the first transformed dataset (i.e., the unlabeled data after the first transformation, X_{A}^{u}), may be performed by utilizing the current machine learning model to obtain the first prediction result Ŷ_{A}^{u} for X_{A}^{u}. Similarly, by means of performing a second prediction of physiological-related parameter on the first transformed dataset (i.e:, the unlabeled data after the second transformation X_{B}^{u}) using the current machine learning model, a second prediction result Ŷ_{B}^{u} may be obtained for X_{B}^{u}.

Next, during training process on the machine learning model, a difference between the first prediction result and the second prediction result may be used as a loss term, which may be noted as [symbol] ^{u}, e.g., squared L₂ norm loss ∥Ŷ_{A}^{u}−Ŷ_{B}^{u}∥₂², where Ŷ_{A}^{u}=Ø(X_{A}^{u}; θ′), and Ŷ_{B}^{u}=Ø(X_{B}^{u}; θ′).

In some embodiments, regression loss on the clean dataset L_{c} can be used together with this loss term [symbol] ^{u} on the unlabeled dataset to train a higher quality model via back-propagation algorithms. Based on joint loss terms containing both these loss terms, a better training (retraining) may be performed on the machine learning model to improve the performance of the machine learning model for more accurate prediction results in the test stage for the physiological-related parameter prediction.

Additionally, in some embodiments, when performing training of the machine learning model using the data in the unlabeled dataset [symbol] ᵤ, the prior information associated with the data in the unlabeled dataset [symbol] ᵤ may also be used to perform data labeling or generate additional loss item. For example, the prior information can be used to generate pseudo-labels for the unlabeled or weakly-labeled data. The prior information may include at least one of the following and/or a combination thereof: a label that a FFR. value at ostia point is labeled as 1 or indicates a maximum value, data of a vessel without lesion is labeled to indicate normality value or normal label, data of a vessel with first stenosis degree or more severe stenosis is labeled to indicate functional significant value or label.

Taking the FFR or iFR prediction task as example, healthy vessels without lesion could be considered as normal ones but vessels with severe stenosis (for example larger than 90% occlusion) could be regarded as functional significant vessels. In addition, FFR values at ostia point may be assumed as 1 based on the definition of FFR. Thus, a large number of images or images of different patients with prior information may be provided during training to boost the performance of the machine learning model. The second dataset may have no invasive measurements or few ones at sparse locations. Additional loss item could be added based on such measurements for the model training.

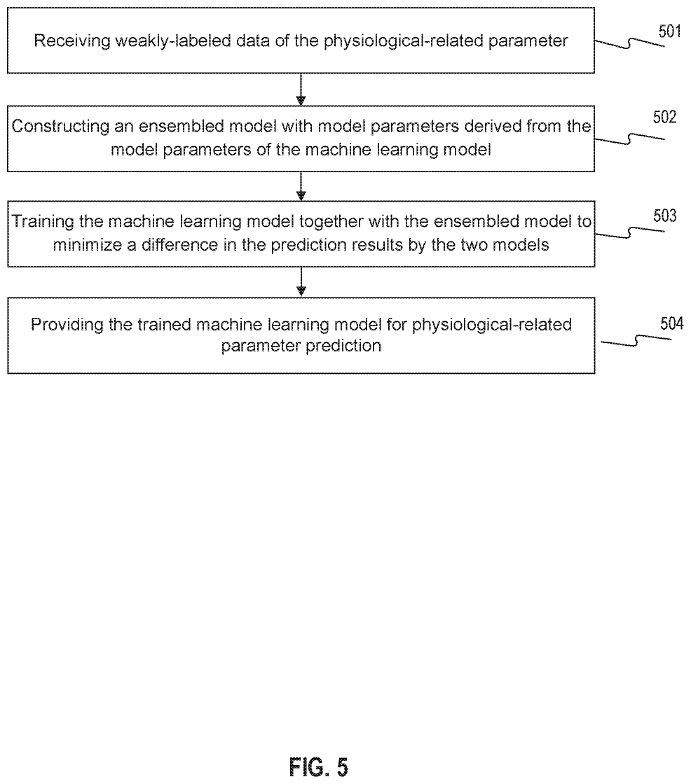

FIG. 5 illustrates flowchart of a third exemplary training method of a machine learning model for physiological-related parameter prediction, according to an embodiment of the present disclosure.

In step 501, weakly-labeled data of the physiological-related parameter may be received. As described foregoing, the above weakly-labeled data may include at least one of the following image data from plurality of data sources and/or a combination thereof: functional MRI, Cone Beam CT (CBCT), Spiral CT, Positron Emission Tomography (PET), Single-Photon Emission Computed Tomography (SPECT), X-ray, optical tomography, fluorescence imaging, ultrasound imaging, and radiotherapy portal imaging and so on, which is not repeated here.

In step 502, one or more ensemble machine learning model(s) may be constructed with model parameters derived from the model parameters of the machine learning model. That is, the machine learning model may have a first set of model parameters that define it, and each ensemble model may have a second set of model parameters that are derived from the first set of model parameters. In some embodiments, the model parameters of the ensemble model may be a moving average of historical values of the model parameters of the machine learning models throughout the training process. For example, the historical values of the model parameters are the historical weights of the machine learning model at different training steps.

In step 503, the machine learning model and the one or more ensemble models are trained together in a way to minimize the difference in prediction results by these models. For example, a first prediction may be perfon ied on the weakly-labeled data by utilizing the machine learning model, and a second prediction may be performed on the weakly-labeled data by utilizing the ensemble machine learning model, and a difference between the first prediction result and the second prediction result may be used as a loss term to regulate the training.

In step 504, the trained machine learning model may be used for physiological-related parameter prediction.

In some embodiments, like the second exemplary training method, the training of the third exemplary method can also be assisted by using a loss term formulated with the clean labeled data in the received training data. In yet some embodiments, prior infoiination derived from the weakly-labeled data can be further used to facilitate the training. The prior information may include at least one of the following and/or a combination thereof: a label that a FFR value at ostia point is labeled as 1 or indicates a maximum value, data of a vessel without lesion is labeled to indicate normality value or normal label, data of a vessel with first stenosis degree or more severe stenosis is labeled to indicate functional significant value or label.

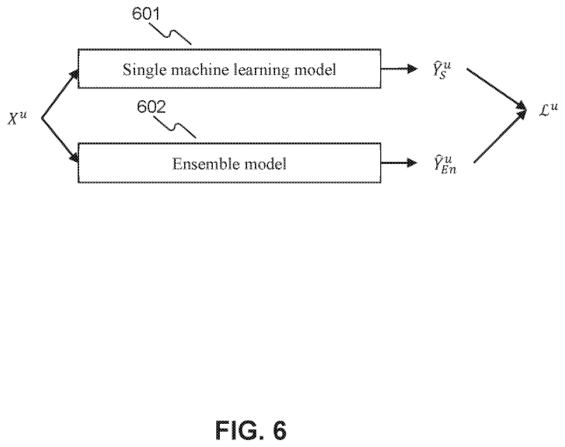

FIG. 6 illustrates a schematic diagram of the third exemplary training method of FIG. 5, according to an embodiment of the present disclosure.

As shown in FIG. 6, a single machine learning model 601 0(1.; 0) is to be trained by the third exemplary training method. The third exemplary training method leverages the dfference(s) between the predictions of one or more ensembled models and the single model to assist the training. The assumption is that the performance of the enseinbled model is usually better than the single. Thus, the ensemble model can be used to supervise the training of the model.

As shown in FIG. 6, an ensemble model 602 Ø(·; θ′ₜ) may be constructed with model parameters derived from those of the single machine learning model 601. In some embodiments, the ensemble model 602 may be derived from a plurality of historical versions of machine learning model {Ø(·; θ₁), . . . , Ø(·; θₜ)} generated during the training process, where Ø(·; θₜ)may be the generated model in the training step t In one example, the ensemble model Ø(·; θ^{′}ₜ) may be generated as follows: selecting several models with relative high performance out of {Ø(·; θ₁), . . . , Ø(·; θₜ)}, then taking the weighted average of the selected models as the ensemble model. In another example, a moving average of the historical versions of single machine learning model {Ø(·; θ₁), . . . , Ø(·; θₜ)} may be adopted as the ensemble model 602 Ø(·; θ^{′}ₜ), where θ^{′}ₜ=αθ^{′}ₜ₋₁+(1−αθₜ) may be the moving average of the history weights of the single machine learning model 601, where θₜ is the single machine learning model's weight at training step t, and αindicates the ensembling weight parameter, which may be used to adjust the degree of model ensembling (or, the degree of model fusing).

With the prior knowledge that the performance of the ensemble machine learning model Ø(·; θ′ₜ) is usually better than that of the single machine learning model Ø(·; θ), the ensemble model may be used to perform supervised training of the model, e.g., training the machine learning model by utilizing the divergence between the prediction results of the ensemble model and the single model.

Specifically, as shown in FIG. 6, a first prediction may be performed on the unlabeled data X^{u} in the dataset [symbol] ᵤ, by utilizing the single machine learning model 601, yielding the first prediction result Ŷ_{S}^{u}.

Similarly, a second prediction may be performed on the unlabeled data X^{u} in the dataset [symbol] ᵤ , by utilizing the ensemble model 602 Ø(·; θ′ₜ), yielding he second prediction result Ŷ_{En}^{u}.

Based on Ŷ_{S}^{u} and Ŷ_{En}^{u}, the first loss item [symbol] ^{u} may be generated, e.g., squared L₂ norm loss ∥Ŷ_{En}^{u}−Ŷ_{S}^{u}∥₂², where Ŷ_{En}^{u}=Ø(X_{En}^{u}; θ′), and Ŷ_{S}^{u}=Ø(X_{S}^{u}; θ).

The training may be perfau led on the single machine learning model 601 together with the ensemble model 602 by utilizing a loss function containing this first loss term [symbol] ^{u}.

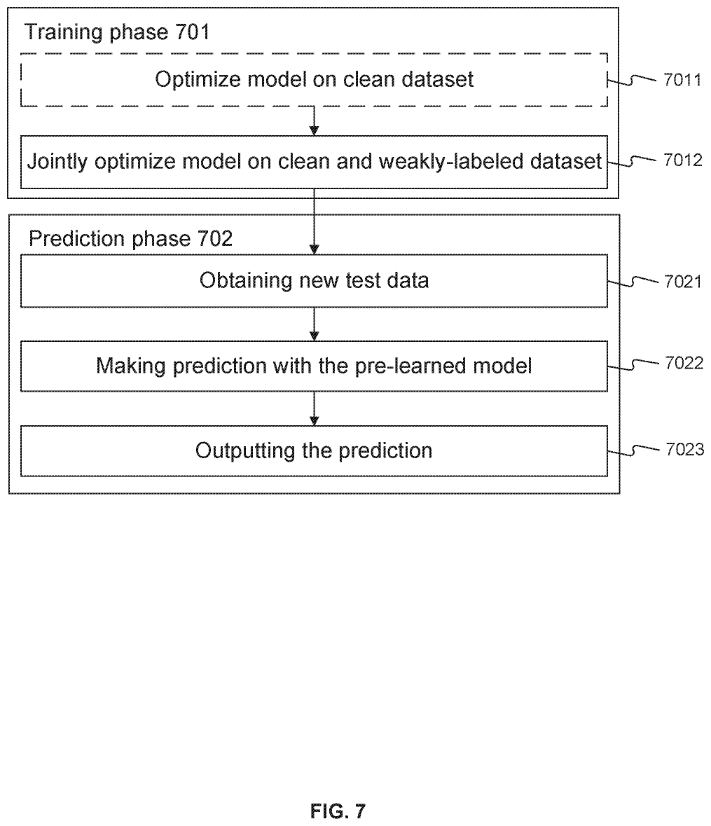

In other embodiments, the first loss term [symbol] ^{u} and a second loss term L_{c} may be taken into account altogether. The second loss term L_{c} indicates a difference between the prediction result Ŷ^{c} by the single machine learning model 601 and ground truth Y^{c}. The detailed procedure and algorithm of the calculation of L_{c} has been described by referring to FIG. 2, and thus are not repeated here. FIG. 7 illustrates a flowchart of the training and testing process of physiological-related parameter prediction using training data including labeled data and weakly-labeled data, according to an embodiment of the present disclosure.

As shown in FIG. 7, the training phase and prediction phase of the machine learning model for physiological-related parameter prediction on training data including both the labeled dataset (e.g., clean dataset) and weakly-labeled dataset may be performed as follows.

In some embodiments, the training phase 701 may be an offline process. Step 7011 may be an optional step, which aiming to learn a mapping between the inputs and the ground truth by finding the best fit between predictions and ground truth values over the clean dataset. For example, step 7011 can be performed according to step 102 in the first exemplary training method. In step 7012, the model may be trained or refined (if step 7011 is performed) on the clean and weakly-labeled datasets, jointly. For example, step 7012 can be performed according to step 104 in the first exemplary training method, or according to the second or third exemplary training method described above. The weakly-labeled dataset may be used to boost the model performance. The ground truth might be available for all positions, or partial segments or even some locations in sequences. The ground truth could be single value for one position, or it could be multiple values (e.g., vector, matrix, tensor, and so on) for one position.

In some embodiments, the prediction phase 702 may be an online process, whereby the predictions for an unseen data are calculated by using the learned mapping from the training phase 701. Particularly, the prediction phase 602 may be divided into three steps as follows.

In step 7021, new test data, e.g., newly acquired image data, may be received for prediction.

In step 7022, prediction may be performed on the test data, with the machine learning model obtained by the training phase 701, to generate prediction result.

In step 7023, the prediction result may be output. Particularly, the prediction result may be presented, in visual and/or audible manners, to inform the user or provide prompts to the user.

The disclosed system and method of training the machine learning model for physiological-related parameter prediction according to any embodiment of the present disclosure may be applied or adapted to train machine learning models by using weakly-labeled or unlabeled data acquired in different context by different means, to predict various different medical or physiological-related parameters, including but not limited to FFR or iFR prediction task.

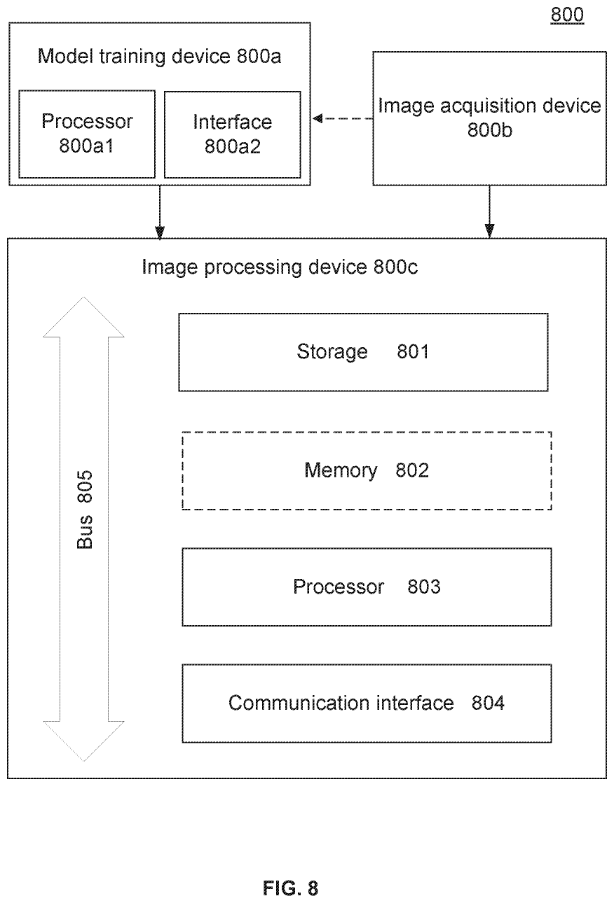

FIG. 8 illustrates a schematic block diagram of a training system of the machine learning model for physiological-related parameter prediction, according to the embodiment of the present disclosure.

As shown in FIG. 8, the training system may include a model training device 800a, an image acquisition device 800b and an image analysis device 800c.

The systems may include a model training device 800a configured to perfoiirr the training method according to any embodiment of present disclosure (e.g., the offline training phase shown as training phase 701 in FIG. 7) and an image processing device 700c configured to perform the prediction process by using the machine learning model obtained at any training step of the training method as above (e.g., the online prediction phase shown as prediction phase in FIG. 7).

In some embodiments, model training device 800a and image processing device 800c may be inside the same computer or processing device.

In some embodiments, image processing device 800c may be a special-purpose computer, or a general-purpose computer. For example, image processing device 800c may be a computer custom-built for hospitals to perform image acquisition and image processing tasks, or a server placed in the cloud. Image processing device 800c may include a communication interface 804, a storage 801, a memory 802, a processor 803, and a bus 805. Communication interface 804, storage 801, memory 802, and processor 803 are connected with bus 805 and communicate with each other through bus 805.

Communication interface 804 may include a network adaptor, a cable connector, a serial connector, a USB connector, a parallel connector, a high-speed data transmission adaptor, such as fiber, USB 3.0, thunderbolt, and the like, a wireless network adaptor, such as a WiFi adaptor, a telecommunication (3G, 4G/LTE and the like) adaptor, etc. In some embodiments, communication interface 804 receives biomedical images (each including a sequence of image slices) from image acquisition device 800b. In some embodiments, communication interface 804 also receives the trained learning model from model training device 800a.

Image acquisition device 800b can acquire images of any imaging modality among functional MRI (e.g., fMRI, DCE-MRI and diffusion MRI), Cone Beam CT (CBCT), Spiral CT, Positron Emission Tomography (PET), Single-Photon Emission Computed Tomography (SPECT), X-ray, optical tomography, fluorescence imaging, ultrasound imaging, and radiotherapy portal imaging, etc., or the combination thereof. The disclosed methods can be performed by the system to make various predictions (e.g., FFR predictions) using the acquired images.

Storage 801/memory 802 may be a non-transitory computer-readable medium, such as a read-only memory (ROM), a random access memory (RAM), a phase-change random access memory (PRAM), a static random access memory (SRAM), a dynamic random access memory (DRAM), an electrically erasable programmable read-only memory (EEPROM), other types of random access memories (RAMS), a flash disk or other forms of flash memory, a cache, a register, a static memory, a compact disc read-only memory (CD-ROM), a digital versatile disc (DVD) or other optical storage, a cassette tape or other magnetic storage devices, or any other non-transitory medium that may be used to store information or instructions capable of being accessed by a computer device, etc.

In some embodiments, storage 801 may store the trained learning model and data, such as feature maps generated while executing the computer programs, etc. In some embodiments, memory 802 may store computer-executable instructions, such as one or more image processing programs. In some embodiments, feature maps may be extracted at different granularities from image slices stored in storage 801. The feature maps may be read from storage 801 one by one or simultaneously and stored in memory 802.

Processor 803 may be a processing device that includes one or more general processing devices, such as a microprocessor, a central processing unit (CPU), a graphics processing unit (GPU), and the like. More specifically, the processor may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. The processor may also be one or more dedicated processing devices such as application specific integrated circuits (ASICs), field programmable gate arrays (FPGAs), digital signal processors (DSPs), system-on-chip (SoCs), and the like. Processor 803 may be communicatively coupled to memory 802 and configured to execute the computer-executable instructions stored thereon.

The model training device 7800a may be implemented with hardware specially programmed by software that performs the training process. For example, the model training device 800a may include a processor 800a1 and a non-transitory computer-readable medium (not shown) similar to image processing device 800c. The processor 800a1 may conduct the training by performing instructions of a training process stored in the computer-readable medium. The model training device 800a may additionally include input and output interfaces 800a2 to communicate with training database, network, and/or a user interface. The user interface may be used for selecting sets of training data, adjusting one or more parameters of the training process, selecting or modifying a framework of the learning model, and/or manually or semi-automatically providing prediction results associated with a sequence of images for raining.

Another aspect of the present disclosure is to provide a non-transitory computer readable medium storing instruction thereon, and when implemented, it causes one or more processors to perform the above method. The computer-readable medium may include volatile or nonvolatile, magnetic, semiconductor-based, tape-based, optical, removable, non-removable or other types of computer-readable media or computer-readable storage devices. For example, the computer-readable medium may be a storage device or a storage module in which a computer instruction is stored, as disclosed. In some embodiments, the computer-readable medium may be a magnetic disk or a flash drive on which computer instructions are stored.

Various modifications and changes can be made to the method and system of the present disclosure. Other embodiments can be derived by those skilled in the art in view of the description and practice of the disclosed system and the related method. Each claim of the present disclosure can be understood as an independent embodiment, and any combination between them is also used as an embodiment of the present disclosure, and these embodiments are deemed to be included in the present disclosure.

The description and examples are intended to be exemplary only, and the e scope is indicated by the appended claims and their equivalents.

What is claimed is:

1. A training method for a machine learning model for physiological analysis, comprising:
receiving training data comprising a first dataset of labeled data of a physiological-related parameter and a second dataset of weakly-labeled data of the physiological-related parameter;
training, by at least one processor, an initial machine learning model using the first dataset;
applying, by the at least one processor, the initial machine learning model to the second dataset to generate a third dataset of pseudo-labeled data of the physiological-related parameter;
training, by the at least one processor, the machine learning model based on the first dataset and the third dataset; and
providing the trained machine learning model for predicti he physiological-related parameter.

2. The training method of claim 1, wherein applying the initial machine learning model to the second dataset to generate the third dataset of pseudo-labeled data of the physiological-related parameter further comprises:
predicting the physiological-related parameter for at least a subset of the weakly-labeled data in the second dataset using the initial machine learning model; and
labeling the subset of the weakly-labeled data in the second dataset using the prediction result to form the pseudo-labeled data in the third dataset.

3. The training method of claim 2, further comprising:
selecting pseudo-labeled data satisfying a first preset condition at least associated with a confidence level to be included in the third dataset.

4. The training method of claim 1, wherein training the initial machine learning model uses a first regression loss term formulated by the labeled data in the first dataset, and training the machine learning model uses the first regression loss term and a second regression loss term formulated by the pseudo-labeled data in the third dataset.

5. The training method of claim 1, wherein the physiological-related parameter includes at least one of physiological function state, blood pressure, blood velocity, blood flow-rate, wall-surface shear stress, fractional flow reserve (FFR), microcirculation resistance index (IMR), and instantaneous wave-free ratio (iFR) and or a combination thereof.

6. The training method according to claim 1, further comprising:
labeling another subset of the weakly-labeled data in the second dataset using prior information of the physiological-related parameter to form additional pseudo-labeled data in the third dataset,
wherein the prior information of the physiological-related parameter includes at least one of a predetermined FFR value at an ostia point, a vessel without lesion being normal, or a vessel with a first stenosis degree or more severe stenosis being functional significant.

7. A training method for a machine learning model for physiological analysis, comprising:
receiving training data comprising weakly-labeled data of a physiological-related parameter;
perfori ring, by at least one processor, a first transfori ration on the weakly-labeled data to form a first transformed dataset;
performing, by the at least one processor, a second transformationon the weakly-labeled data to for in a second transformed dataset;
training, by the at least one processor, the machine learning model based on the training data, the first transformed dataset and the second transformed dataset, wherein the training minimizes a difference between a first prediction result of the physiological-related parameter obtained by applying the machine learning model to the first transformed dataset and a second prediction result of the physiological-related parameter obtained by applying the machine learning model to the second transformed dataset; and
providing the trained machine learning model for predicting the physiological-related parameter.

8. The training method of claim 7, wherein the difference is a squared L₂ norm loss formulated with the first prediction result and the second prediction result.

9. The training method of claim 7, wherein the physiological-related parameter includes at least one of physiological function state, blood pressure, blood velocity, blood flow-rate, wall-surface shear stress, fractional flow reserve (FFR), microcirculation resistance index (IMR), and instantaneous wave-free ratio (iFR) and or a combination thereof.

10. The training method of claim 9, further comprising:
deriving prior information of the physiological-related parameter from the weakly-labeled data; and
training the machine learning model further based on the prior information of the physiological-related parameter.

11. The training method of claim 10, wherein the prior information of the physiological-related parameter includes at least one of a predetermined FFR value at an ostia point, a vessel without lesion being normal, or a vessel with a first stenosis degree or more severe stenosis being functional significant.

12. The training method of claim 7, wherein the training data further comprises labeled data, wherein training the machine learning model further minimizes a regression loss term formulated using the labeled data.

13. The training method according to claim 7, wherein each of the first transformation and the second transformation includes at least one of rotation, translation or scaling of the weakly-labeled data.

14. The training method according to claim 7, wherein the weakly-labeled data includes image data acquired using at least one of functional MRI, Cone Beam CT (CBCT), Spiral CT, Positron Emission Tomography (PET), Single-Photon Emission Computed Tomography (SPECT), X-ray, optical tomography, fluorescence imaging, ultrasound imaging, or radiotherapy portal imaging.

15. A training method for a machine learning model for physiological analysis, comprising:
receiving training data comprising weakly-labeled data of a physiological-related parameter;
training, by at least one processor, the machine learning model with an ensembled model based on the training data, wherein the machine learning model has a first set of model parameters, wherein the ensembled model has a second set of model parameters derived from the first set of model parameters, wherein the training minimizes a difference between a first prediction result of the physiological-related parameter obtained by applying the machine learning model to the weakly-labeled data and a second prediction result of the physiological-related parameter obtained by applying the ensembled model to weakly-labeled data; and
providing the trained machine learning model for predicting the physiological-related parameter.

16. The training method of claim 15, wherein the second set of model parameters is derived from historical values of the first set of model parameters.

17. The training method of claim 16, wherein the second set of model parameters is a moving average of the historical values of the first set of model parameters.

18. The training method of claim 15, wherein the difference is a squared L₂ norm loss formulated with the first prediction result and the second prediction result.

19. The training method of claim 15, wherein the physiological-related parameter includes at least one of physiological function state, blood pressure, blood velocity, blood flow-rate, wall-surface shear stress, fractional flow reserve (FFR), microcirculation resistance index (IMR), and instantaneous wave-free ratio (iFR) and or a combination thereof.

20. The training method according to claim 19, further comprising:
deriving prior information of the physiological-related parameter from the weakly-labeled data; and
training the machine learning model further based on prior information of the physiological-related parameter,
wherein the prior information of the physiological-related parameter includes at least one of a predetermined FFR value at an ostia point, a vessel without lesion being normal, or a vessel with a first stenosis degree or more severe stenosis being functional significant.
